(19) United States
(12) Patent Application Publication
(10) Pub. No.: US 2025/0204988 A1
(43) Pub. Date: Jun. 26, 2025

(54) FEMORAL BROACH-BASED MEASUREMENT TOOL

(71) Applicants: Smith & Nephew, Inc., Memphis, TN (US); Smith & Nephew Orthopaedics AG, Zug (CH); Smith & Nephew Asia Pacific PTE. Limited, Singapore (SG)

(72) Inventors: Michael D. Hollandsworth, Jr., Memphis, TN (US); Abel Cham Kim, Cordova, TN (US); Kevin Wayne Belew, Hernando, MS (US); Kevin R. Hays, Somerville, TN (US); Michael Scott Bennett, Southaven, MS (US)

(73) Assignees: Smith & Nephew, Inc., Memphis, TN (US); Smith & Nephew Orthopaedics AG, Zug (CH); Smith & Nephew Asia Pacific PTE. Limited, Singapore (SG)

(21) Appl. No.: 18/852,631

(22) PCT Filed: May 9, 2023
(86) PCT No.: PCT/US2023/021428

§ 371 Date: Sep. 30, 2024

Related U.S. Application Data
(60) Provisional application No. 63/344,086, filed on May 20, 2022.

(51) Int. Cl. A61B 34/20; A61B 17/16

(52) U.S. Cl. CPC A61B 34/20; A61B 17/16; A61B 2034/2046

(57) ABSTRACT

Disclosed herein is a system and method for determining the position of a broach tool used in a total hip arthroplasty procedure, the broach still being positioned in the femoral canal of a femur of the patient and the position being determined post-broaching. Also disclosed herein are examples of broach tool adapters used to attach a tracking array to the broach tool, the adapters having a body shape to mate with the geometry of particular brands of broach tools and having a common slot defined therein to accept a tracking array such as to attach the tracking array to the broach tool.

### RELATED APPLICATIONS

This application claims the benefit of U.S. Provisional Patent Application No. 63/344,086, filed May 20, 2022, entitled “Femoral Broach-Based Measurement Tool”, the contents of which are incorporated herein their entirety.

### FIELD OF THE DISCLOSURE

The present disclosure relates generally to orthopedic procedures, for example, total joint replacement procedures, and, in particular, to total hip arthroplasty procedures.

### BACKGROUND

Orthopedic procedures to replace joints, for example, knees and hips, are well known and commonplace in today's society. Such procedures may require removal or reshaping of portions of a bone to accept an orthopedic implant to replace the original joint. For example, during a total hip arthroplasty, a surgeon needs to broach the femoral canal of the patient to accept an implant having a stem portion which is inserted into the femoral canal and a ball-portion of a ball and socket joint, which is joined with a socket portion that is implanted into the hip bone.

Such procedures may be performed with the assistance of a navigated or robotic-assisted surgical platform which may track the position of the femur of the patient with respect to the surgical theater or to the anatomy of the patient, and to provide guidance to the surgeon during the procedure.

Navigated total ship arthroplasty often includes predictive range-of-motion (ROM) capabilities for the entire implant construct that uses femoral version measurements based off broach and stem data. These capabilities require a robust and accurate connection to femoral components so that tracking arrays can be accurately and repeatedly placed during surgical workflows. The size of incisions and lack of exposure to usable anatomy have presented challenges to solutions that are placed in the normal operative area, especially for supine approaches.

Prior art methods supported stem position measurements using tools connected to the stem taper that hold a tracking array. These tools did not measure the orientation of the femoral component and were free to rotate about the taper axis of the femoral stem. This style of connection is not suitable for range-of-motion capabilities because the orientation of the tracking array must be kept constant with respect to the orientation of the femoral component. Orientation of the femoral component was assumed in place of a reliable measurement process.

Other prior art methods utilize hard connections to the femur for measurements of femoral versions that utilize multiple pins driven into the proximal and distal femur. These pin insertions can interrupt workflow and require additional incisions not present in a traditional total hip arthroplasty procedures. Tracking array holders are connected to these pins via holding clamp instrumentation. Additionally, tracking of femoral broaching uses trackers attached to the broach inserter tool.

In yet other prior art methods, enhanced femoral workflow uses custom trial necks which have landmark registration divots so that a pointer probe can register their orientation and position of the specified femoral component. This trial neck, in addition to a checkpoint screw, is used to complete femoral workflow steps. This prior art method requires a pre-operative CT and does not support the direct anterior approach for enhanced femoral workflow for all stem families.

### SUMMARY OF THE DISCLOSURE

The devices and processes described herein provide a combination of track array holders that enable repeatable fixed connections to devices placed in the proximal femur, for example, the femoral canal broach tool. These instrument combinations consist of a tool attached to the connection geometry of a femoral broach and a tracking array holder attached to the anterior or lateral face of the proximal femur via a magnetic connection to a plate fixated with a screw.

There are two features to the disclosed examples. A first feature is directed to a broach array and femoral array may be used in combination to obtain a live measurement of a version of the femur based on the positioning of the stem taper of the broach tool. The broach array may be attached to the broach tool after completion of the broaching process to measure or otherwise determine the position of broach tool with respect to the anatomy of the femur, which has been registered by the femoral array attached to the femur. Thus, the use of the femoral array and broach array in combination provides an improved method of determining the final femoral version by determining the relative positioning of the two arrays.

A second feature of the disclosed examples is directed to, several different examples of a broach adapter used to attach the broach array to the broach tool are provided. This allows use of the broach array with a variety of broach tools from different manufacturers having different geometries.

In a first example, a method for measuring the position of a broach tool in a total hip arthroplasty procedure includes attaching a first tracking array to the femur of a patient, registering the position of the femur using the tracking array, attaching a second tracking array to a broach tool after broaching the femoral canal and determining the position of the broach tool with respect to the femur based on a comparison between position of the first and second tracking arrays.

In the first example, or any other example disclosed herein, the method further includes wherein the first tracking array is attached to the femur via a mounting plate that magnetically engages with a mating plate on a holder for the first tracking array.

In the first example, or any other example disclosed herein, the method further includes wherein the mounting plate allows for multiple orientations of the first tracking array.

In the first example, or any other example disclosed herein, the method further includes wherein the mounting plate is attached to the proximal femur targeting the greater trochanter.

In the first example, or any other example disclosed herein, the method further includes wherein registering the position of the femur includes tracking the first tracking array using a tracking system having one or more sensors to collect real-time position data and further wherein software running on a surgical computer calculates and registers the position of the femur based on the position data.

In the first example, or any other example disclosed herein, the method further includes wherein attaching the second tracking array to the broach tool includes providing a broach adapter attached to the broach tool and attaching the second tracking array to the broach adapter.

In the first example, or any other example disclosed herein, the method further includes wherein the broach adapter is adapted to fit the geometry of the broach tool.

In the first example, or any other example disclosed herein, the method further includes wherein the broach adapter attaches to the broach tool using a spring mechanism.

In the first example, or any other example disclosed herein, the method further includes wherein the broach adapter includes a slot adapted to accept a proximal end of a tracking array holder.

In the first example, or any other example disclosed herein, the method further includes wherein the proximal end of the tracking array holder includes a spring component having a protrusion thereon which engages a depression defined in the slot of the broaching adapter.

In the first example, or any other example disclosed herein, the method further includes wherein comparing the position of the first and second tracking arrays includes using a tracking system having one or more sensors to collect real-time position data of the first and second tracking arrays and calculating the position of the broach tool relative to the registered position of the femur based on the position data.

In the first example, or any other example disclosed herein, the method further includes wherein calculating the position of the broach tool is performed by software running on a surgical computer.

In a second example, a device for adapting a holder for a tracking array to a broach tool includes a body shaped to adapt to the geometry of the broach tool, a lever forced by a spring to engage a feature of the geometry of the broach tool and a slot defined in the body of the device for accepting a holder for a tracking array.

In the second example, or any other example disclosed herein, the device further includes wherein the broach tool is disengaged from the body by pivoting the lever such as to disengage the lever from the feature of the geometry of the broach tool to which it was engaged.

In the second example, or any other example disclosed herein, the device further includes a holder for a tracking array, wherein the slot defined in the body is adapted to accept a proximal end of the holder for the tracking array, the proximal end including an arm having a protrusion defined thereon that engages a depression defined in the slot.

In the second example, or any other example disclosed herein, the device further includes wherein a distal end of the holder for the tracking array includes multiple arms, at least some of the arms defining shoulders to prevent rotation or tilting of the holder with respect to the body.

In the second example, or any other example disclosed herein, the device further includes wherein the holder for the tracking array is disengaged from the body by bending the arm such that the protrusion disengages from the depression defined in the slot.

In a third example, a system includes a tracking system having one or more sensors, a processor and software that, when executed by the processor, causes the system to determine a position of a femur by tracking a first tracking array mechanically connected to the femur using the tracking system to collect real-time position data of the first tracking array and determine the position of a broach tool disposed in the femoral canal of the femur by tracking a second tracking array mechanically connected to the broach tool using the tracking system to collect real-time position data of the second tracking array, wherein the software calculates the position of the broach tool with respect to the femur based on a comparison of the position data representing the position of the first and second tracking arrays.

Examples of the present disclosure provide numerous advantages. For example, by allowing multiple engagements and disengagements of the tracking array during the procedure without the need to re-register the array. In addition, various examples of the broach adapter are provided for broach tools with different geometries, all broach adapters having a common interface for accepting the holder of the broach tracking array such that the broach tracking array may be used with broach tools from different manufacturers.

Further features and advantages of at least some of the examples of the presently disclosed examples, as well as the structure and operation of various examples of the presently disclosed examples, are described in detail below with reference to the accompanying drawings.

### BRIEF DESCRIPTION OF THE DRAWINGS

By way of example, specific examples of the disclosed system and method will now be described, with reference to the accompanying drawings, in which:

FIG. 1 depicts an operating theatre including an illustrative computer-assisted surgical system (CASS) in accordance with the described examples.

FIG. 2 is a depiction of first tracking array attached to the femur of a patient and a second tracking array attached to a broach tool such as to determine the position of the broach tool by comparison of the positions of the tracking arrays.

FIGS. 3A-3D depict various views of the attachment of a mounting plate to the femur of a patient to accept a holder for a tracking array via magnetic engagement.

FIGS. 4A-4C show various views of a first example of a broach adapter.

FIGS. 5A-5C show various views of a second example of the broach adapter.

FIGS. 6A-6C show various views of a third example of a broach adapter.

FIGS. 7A and 7B show details of a proximal end of a tracking array holder engaged with a broach adapter.

### DEFINITIONS

For the purposes of this disclosure, the term “implant” is used to refer to a prosthetic device or structure manufactured to replace or enhance a biological structure. For example, in a total hip replacement procedure a prosthetic acetabular cup (implant) is used to replace or enhance a patients' worn or damaged acetabulum. While the term “implant” is generally considered to denote a man-made structure (as contrasted with a transplant), for the purposes of this specification an implant can include a biological tissue or material transplanted to replace or enhance a biological structure.

Although much of this disclosure refers to surgeons or other medical professionals by specific job title or role, nothing in this disclosure is intended to be limited to a specific job title or function. Surgeons or medical professionals can include any doctor, nurse, medical professional, or technician. Any of these terms or job titles can be used interchangeably with the user of the systems disclosed herein unless otherwise explicitly stated. For example, a reference to a surgeon could also apply, in some examples to a technician or nurse.

### DETAILED DESCRIPTION

The system and method disclosed herein provides information to a surgeon during the pre-operative and intra-operative phases of a joint replacement surgery which will assist the surgeon in planning and executing the surgery such as to minimize both intra-operative and post-operative negative outcomes for the patient. Note that, although the disclosed examples are explained in terms of a total hip replacement procedure, all or portions of the disclosed examples may be applicable to any joint replacement procedure.

This disclosure is not limited to the particular systems, devices and methods described, as these may vary. The terminology used in the description is for the purpose of describing the particular versions or examples only and is not intended to limit the scope.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. As used in this document, the term “comprising” means “including, but not limited to”.

### CASS Overview

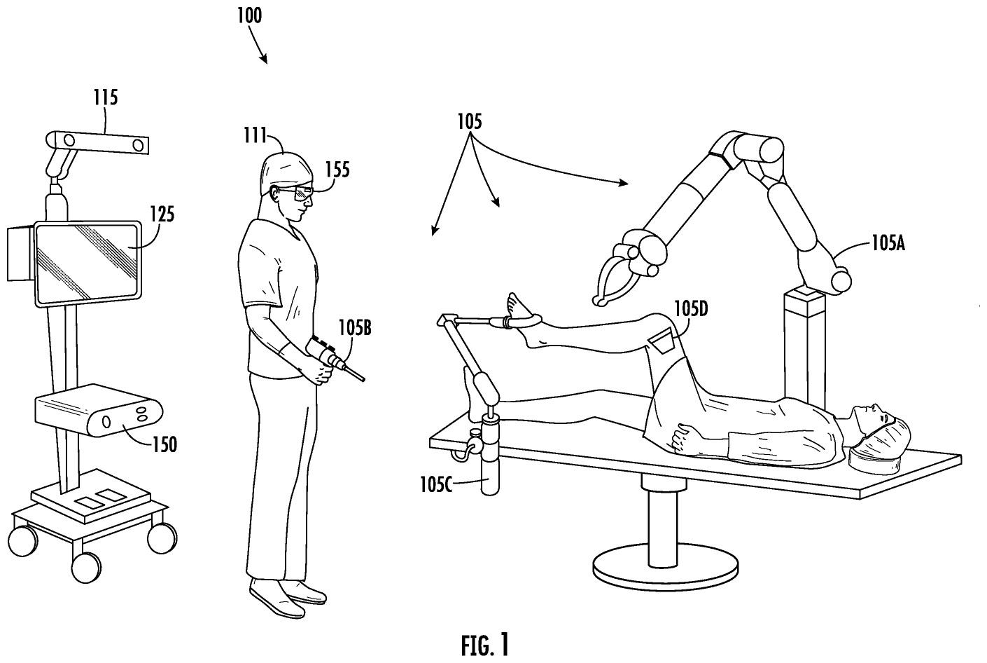

FIG. 1 provides an illustration of an example computer-assisted surgical system (CASS) 100, according to some examples. As described in further detail in the sections that follow, the CASS uses computers, robotics, and imaging technology to aid surgeons in performing orthopedic surgery procedures such as total knee arthroplasty (TKA) or total hip arthroplasty (THA). For example, surgical navigation systems can aid surgeons in locating patient anatomical structures, guiding surgical instruments, and implanting medical devices with a high degree of accuracy. Surgical navigation systems such as the CASS 100 often employ various forms of computing technology to perform a wide variety of standard and minimally invasive surgical procedures and techniques. Moreover, these systems allow surgeons to more accurately plan, track and navigate the placement of instruments and implants relative to the body of a patient, as well as conduct pre-operative and intra-operative body imaging.

An effector platform 105 positions surgical tools relative to a patient during surgery. The exact components of the effector platform 105 will vary, depending on the example employed. For example, for a knee surgery, the effector platform 105 may include an end effector 105B that holds surgical tools or instruments during their use. The end effector 105B may be a handheld device or instrument used by the surgeon (e.g., a NAVIO® hand piece or a cutting guide or jig) or, alternatively, the end effector 105B can include a device or instrument held or positioned by a robotic arm 105A.

The effector platform 105 can include a limb positioner 105C for positioning the patient's limbs during surgery. One example of a limb positioner 105C is the SMITH AND NEPHEW SPIDER2 system. The limb positioner 105C may be operated manually by the surgeon or alternatively change limb positions based on instructions received from the surgical computer 150 (described below).

The effector platform 105 can also include a cutting guide or jig 105D that is used to guide saws or drills used to resect tissue during surgery. Such cutting guides 105D can be formed integrally as part of the effector platform 105 or robotic arm 105A or cutting guides can be separate structures that can be matingly and/or removably attached to the effector platform 105 or robotic arm 105A. The effector platform 105 or robotic arm 105A can be controlled by the CASS 100 to position a cutting guide or jig 105D adjacent to the patient's anatomy in accordance with a pre-operatively or intraoperatively developed surgical plan such that the cutting guide or jig will produce a precise bone cut in accordance with the surgical plan.

The tracking system 115 uses one or more sensors to collect real-time position data that locates the patient's anatomy and surgical instruments. For example, for TKA procedures, the tracking system may provide a location and orientation of the end effector 105B during the procedure. In addition to positional data, data from the tracking system 115 can also be used to infer velocity/acceleration of anatomy/instrumentation, which can be used for tool control. In some examples, the tracking system 115 may use a tracker array attached to the end effector 105B to determine the location and orientation of the end effector 105B. The position of the end effector 105B may be inferred based on the position and orientation of the tracking system 115 and a known relationship in three-dimensional space between the tracking system 115 and the end effector 105B. Various types of tracking systems may be used in various examples of the presently disclosed examples including, without limitation, Infrared (IR) tracking systems, electromagnetic (EM) tracking systems, video or image based tracking systems, and ultrasound registration and tracking systems.

Any suitable tracking system can be used for tracking surgical objects and patient anatomy in the surgical theatre. For example, a combination of IR and visible light cameras can be used in an array. Various illumination sources, such as an IR LED light source, can illuminate the scene allowing three-dimensional imaging to occur. In some examples, this can include stereoscopic, tri-scopic, quad-scopic, etc. imaging. In addition to the camera array, which in some examples is affixed to a cart, additional cameras can be placed throughout the surgical theatre. For example, handheld tools or headsets worn by operators/surgeons can include imaging capability that communicates images back to a central processor to correlate those images with images captured by the camera array. This can give a more robust image of the environment for modeling using multiple perspectives. Furthermore, some imaging devices may be of suitable resolution or have a suitable perspective on the scene to pick up information stored in quick response (QR) codes or barcodes. This can be helpful in identifying specific objects not manually registered with the system.

In some examples, specific objects can be manually registered by a surgeon with the system preoperatively or intraoperatively. For example, by interacting with a user interface, a surgeon may identify the starting location for a tool or a bone structure. By tracking fiducial marks associated with that tool or bone structure, or by using other conventional image tracking modalities, a processor may track that tool or bone as it moves through the environment in a three-dimensional model.

In some examples, certain markers, such as fiducial markers that identify individuals, important tools, or bones in the theater may include passive or active identifiers that can be picked up by a camera or camera array associated with the tracking system. For example, an IR LED can flash a pattern that conveys a unique identifier to the source of that pattern, providing a dynamic identification mark. Similarly, one or two dimensional optical codes (barcode, QR code, etc.) can be affixed to objects in the theater to provide passive identification that can occur based on image analysis. If these codes are placed asymmetrically on an object, they can also be used to determine an orientation of an object by comparing the location of the identifier with the extents of an object in an image. For example, a QR code may be placed in a corner of a tool tray, allowing the orientation and identity of that tray to be tracked. Other tracking modalities are explained throughout. For example, in some examples, augmented reality headsets can be worn by surgeons and other staff to provide additional camera angles and tracking capabilities.

In addition to optical tracking, certain features of objects can be tracked by registering physical properties of the object and associating them with objects that can be tracked, such as fiducial marks fixed to a tool or bone. For example, a surgeon may perform a manual registration process whereby a tracked tool and a tracked bone can be manipulated relative to one another. By impinging the tip of the tool against the surface of the bone, a three-dimensional surface can be mapped for that bone that is associated with a position and orientation relative to the frame of reference of that fiducial mark. By optically tracking the position and orientation (pose) of the fiducial mark associated with that bone, a model of that surface can be tracked with an environment through extrapolation.

The registration process that registers the CASS 100 to the relevant anatomy of the patient can also involve the use of anatomical landmarks, such as landmarks on a bone or cartilage. For example, the CASS 100 can include a 3D model of the relevant bone or joint and the surgeon can intraoperatively collect data regarding the location of bony landmarks on the patient's actual bone using a probe that is connected to the CASS. Bony landmarks can include, for example, the medial malleolus and lateral malleolus, the ends of the proximal femur and distal tibia, and the center of the hip joint. The CASS 100 can compare and register the location data of bony landmarks collected by the surgeon with the probe with the location data of the same landmarks in the 3D model. Alternatively, the CASS 100 can construct a 3D model of the bone or joint without pre-operative image data by using location data of bony landmarks and the bone surface that are collected by the surgeon using a CASS probe or other means. The registration process can also include determining various axes of a joint. For example, for a TKA the surgeon can use the CASS 100 to determine the anatomical and mechanical axes of the femur and tibia. The surgeon and the CASS 100 can identify the center of the hip joint by moving the patient's leg in a spiral direction (i.e., circumduction) so the CASS can determine where the center of the hip joint is located.

The display 125 provides graphical user interfaces (GUls) that display images and other information relevant to the surgery. For example, the display 125 overlays image information collected from various modalities (e.g., CT, MRI, X-ray, fluorescent, ultrasound, etc.) collected pre-operatively or intraoperatively to give the surgeon various views of the patient's anatomy as well as real-time conditions. The display 125 may include, for example, one or more computer monitors. As an alternative or supplement to the display 125, one or more members of the surgical staff may wear an augmented reality (AR) head mounted device (HMD). For example, in FIG. 1 the surgeon 111 is wearing an AR HMD 155 that may, for example, overlay pre-operative image data on the patient or provide surgical planning suggestions. Various example uses of the AR HMD 155 in surgical procedures are detailed in the sections that follow.

Surgical computer 150 provides control instructions to various components of the CASS 100, collects data from those components, and provides general processing for various data needed during surgery. In some examples, the surgical computer 150 is a general purpose computer. In other examples, the surgical computer 150 may be a parallel computing platform that uses central processing units (CPUs), graphics processing units (GPU), tensor processing units (TPU) or multiple computer instances in a cluster to perform processing. In some examples, the surgical computer 150 is connected to a remote server over one or more computer networks (e.g., the Internet). The remote server can be used, for example, for storage of data or execution of computationally intensive processing tasks.

Various techniques generally known in the art can be used for connecting the surgical computer 150 to the other components of the CASS 100. Moreover, the computers can connect to the surgical computer 150 using a mix of technologies. For example, the end effector 105B may connect to the surgical computer 150 over a wired (i.e., serial) connection. The tracking system 115, tissue navigation system 120, and display 125 can similarly be connected to the surgical computer 150 using wired connections. Alternatively, the tracking system 115, tissue navigation system 120, and display 125 may connect to the surgical computer 150 using wireless technologies such as, without limitation, Wi-Fi, Bluetooth, Near Field Communication (NFC), or ZigBee.

In some examples, CASS 100 may include a robotic arm 105A that serves as an interface to stabilize and hold a variety of instruments used during the surgical procedure. For example, in the context of a hip surgery, these instruments may include, without limitation, retractors, a sagittal or reciprocating saw, the reamer handle, the cup impactor, the broach handle, and the stem inserter. The robotic arm 105A may have multiple degrees of freedom (like a Spider device) and have the ability to be locked in place (e.g., by a press of a button, voice activation, a surgeon removing a hand from the robotic arm, or other method).

In some examples, movement of the robotic arm 105A may be effectuated by use of a control panel built into the robotic arm system. For example, a display screen may include one or more input sources, such as physical buttons or a user interface having one or more icons, that direct movement of the robotic arm 105A. The surgeon or other healthcare professional may engage with the one or more input sources to position the robotic arm 105A when performing a surgical procedure.

A tool or an end effector 105B, for example, a powered surgical tool, attached or integrated into a robotic arm 105A may include, without limitation, a burring device, a scalpel, a cutting device, a retractor, a joint tensioning device, or the like. In examples in which an end effector 105B is used, the end effector may be positioned at the end of the robotic arm 105A such that any motor control operations are performed within the robotic arm system. In examples in which a tool is used, the tool may be secured at a distal end of the robotic arm 105A, but motor control operation may reside within the tool itself.

The robotic arm 105A may be motorized internally to both stabilize the robotic arm, thereby preventing it from falling and hitting the patient, surgical table, surgical staff, etc., and to allow the surgeon to move the robotic arm without having to fully support its weight. While the surgeon is moving the robotic arm 105A, the robotic arm may provide some resistance to prevent the robotic arm from moving too fast or having too many degrees of freedom active at once. The position and the lock status of the robotic arm 105A may be tracked, for example, by a controller or the Surgical Computer 150.

In some examples, the robotic arm 105A can be moved by hand (e.g., by the surgeon) or with internal motors into its ideal position and orientation for the task being performed. In some examples, the robotic arm 105A may be enabled to operate in a “free” mode that allows the surgeon to position the arm into a desired position without being restricted. While in the free mode, the position and orientation of the robotic arm 105A may still be tracked as described above. In some examples, certain degrees of freedom can be selectively released upon input from user (e.g., surgeon) during specified portions of the surgical plan tracked by the Surgical Computer 150. Designs in which a robotic arm 105A is internally powered through hydraulics or motors or provides resistance to external manual motion through similar means can be described as powered robotic arms, while arms that are manually manipulated without power feedback, but which may be manually or automatically locked in place, may be described as passive robotic arms.

A robotic arm 105A or end effector 105B can include a trigger or other means to control the power of a saw or drill. Engagement of the trigger or other means by the surgeon can cause the robotic arm 105A or end effector 105B to transition from a motorized alignment mode to a mode where the saw or drill is engaged and powered on. Additionally, the CASS 100 can include a foot pedal (not shown) that causes the system to perform certain functions when activated. For example, the surgeon can activate the foot pedal to instruct the CASS 100 to place the robotic arm 105A or end effector 105B in an automatic mode that brings the robotic arm or end effector into the proper position with respect to the patient's anatomy in order to perform the necessary resections. The CASS 100 can also place the robotic arm 105A or end effector 105B in a collaborative mode that allows the surgeon to manually manipulate and position the robotic arm or end effector into a particular location. The collaborative mode can be configured to allow the surgeon to move the robotic arm 105A or end effector 105B medially or laterally, while restricting movement in other directions. As discussed, the robotic arm 105A or end effector 105B can include a cutting device (saw, drill, and burr) or a cutting guide or jig 105D that will guide a cutting device. In other examples, movement of the robotic arm 105A or robotically controlled end effector 105B can be controlled entirely by the CASS 100 without any, or with only minimal, assistance or input from a surgeon or other medical professional. In still other examples, the movement of the robotic arm 105A or robotically controlled end effector 105B can be controlled remotely by a surgeon or other medical professional using a control mechanism separate from the robotic arm or robotically controlled end effector device, for example using a joystick or interactive monitor or display control device.

### DESCRIPTION OF EXAMPLES

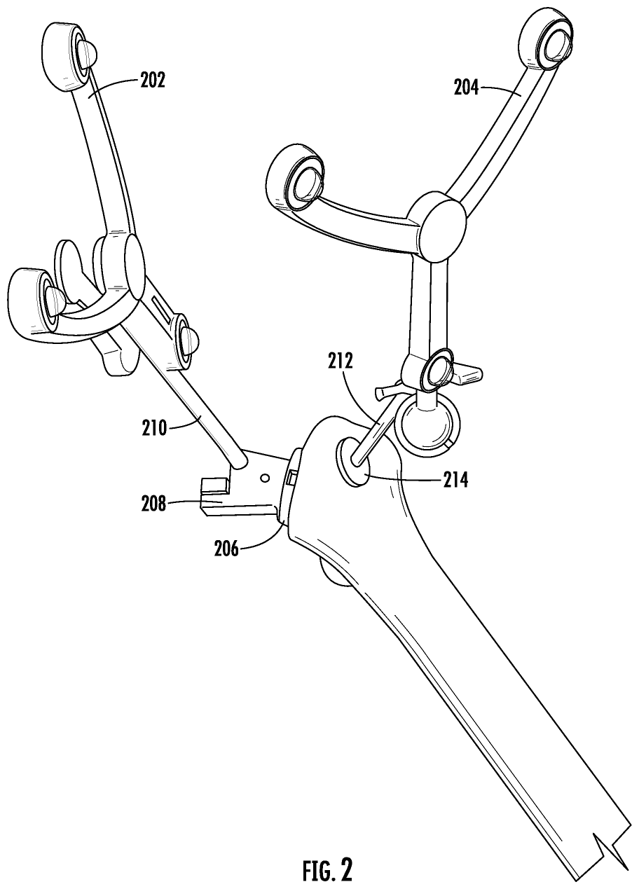

A first feature of the disclosed examples, shown in FIG. 2, is directed to a method wherein a broach array 202 and a femoral array 204 may be used together to determine the positioning of the broach tool 206 within the femoral canal. The femoral array 204, which is attached to the femur in various positions depending upon the surgical approach used, establishes a frame of reference in which the femoral anatomy is registered. The positioning of the broach tool 206 is performed by detecting the positioning of the broach array 202, which is connected to broach tool 206 via broach adapter 208, with respect to the frame of reference established by the femoral array 204.

In various disclosed examples, the femoral array 204 and broach array 202 may be tracked by sensors which are part of tracking system 115. Tracking system 115 may be in communication with surgical computer 150 which may take array tracking data from tracking system 115 and display the positioning of broach tool 206 with respect to the femoral canal on display 125. The frame of reference may be established by software executing on surgical computer 153 pre-broaching by scanning the position of the femoral array 204 with respect to the surgical theater and registering the femoral anatomy. Post-broaching, the positioning of the femoral broach tool 206 may be calculated by the software executing on surgical computer 150 by receiving data indicating the positioning of the broach array 202 which may be compared to the femoral anatomy to determine the position of the broach tool 206 within the femoral canal. The final positioning of the broach implant (not shown) may be inferred by the determined position of the broach tool 206.

Broach adapter 208 is configured to mate with the geometry of the upper portion of broach tool 206. The broach array 202 is connected to the broach adapter 208 via array holder 210, which fits into a slot on broach adapter 208, as will be described later. The femoral array 204 is connected to the femur via array holder 212 which is configured with a plate 214 that is magnetically attached mating plate attached to the femur, as will be described later.

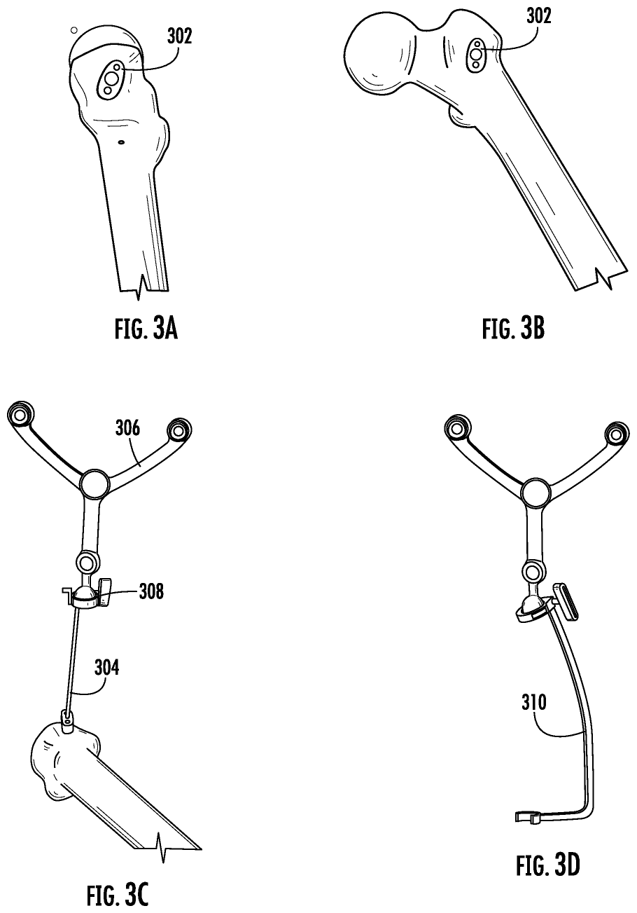

FIGS. 3A-3D show various methods, in accordance with the present disclosed examples, of attaching the femoral array to the femur of the patient. Shown in FIGS. 3A-3D are different methods of orienting an in-wound plate used for attaching the femoral array holder and femoral array to the femur. For differing surgical approaches, for example, a lateral approach shown in FIG. 3A and a supine approach shown in FIG. 3B, the attachment plate 302 may be attached to the femur in different places. In one approach, the in-wound plate 302 is placed on the proximal femur targeting the greater trochanter. A small osteopenia screw is placed through a center hole in attachment plate 302 to fully fixate attachment plate 302 to the femur.

The array holder 304, shown in FIG. 3C uses a patching pin geometry and encapsulated magnet to magnetically engage array holder 304 with attachment plate 302. The magnetic connection creates a stiff construct that can be left on while positioning the femur and completing workflow steps required to broach the femoral canal and seat the femoral implant into the femur. The magnetic connection allows removal of array 306 and reattachment in the same position to avoid having to reregister the array 306 each time it is removed. In various examples, the attachment plate 302 may have multiple facets to allow for multiple orientations of array holder 304. For example, attachment plate may allow for 4 connection orientations spaced at 90° intervals. Other numbers of orientations are also contemplated to be within the scope of the disclosed examples. The particular facet that the array holder 304 is attached to may be determined and communicated to software executing on surgical computer 150 to allow for correct calculation of the relative positioning of the orientation of the array holder 304.

The femoral array 306 may be fixed using a thumbscrew 308 at the top of array holder 304. The position of array 306 is maintained throughout the surgery to establish references for the femur pre- and post-femoral workflow steps. Array holder 304 may be configured in different ways to accommodate different surgical procedures. For example, FIG. 3D shows a curved array holder 310.

Total hip arthroplasty procedures may be accomplished using femoral implants and broach tools made by different manufacturers. The femoral broaches made by each manufacturer may have different geometries for attachment of tools to the top portion of the broach, for example, an impact tool to force the broach tool into the femoral canal. Therefore, a second feature of the disclosed examples is directed to providing different broach adapters configured to adapt to broach tools with different geometries, to allow attachment of the broach array securely to the top portion of the femoral broach tool, post-broaching, to determine the positioning of the broach tool with respect to the femur. As previously described, the position can be determined by tracking the relative positions of the broach array and the femoral array.

There are three current examples of a broach adapter disclosed herein that vary by compatible stem families. Each broach adapter utilizes a mechanism that latches onto a broach connection geometry located at the proximal end of the broach tool. The mechanisms disclosed herein may mimic the functionality and geometry of trial neck devices. Broach arrays are connected to the broach adapters via a spring tab mechanism inserted into a unique slot geometry that is shared by in common to the broach adapters disclosed herein.

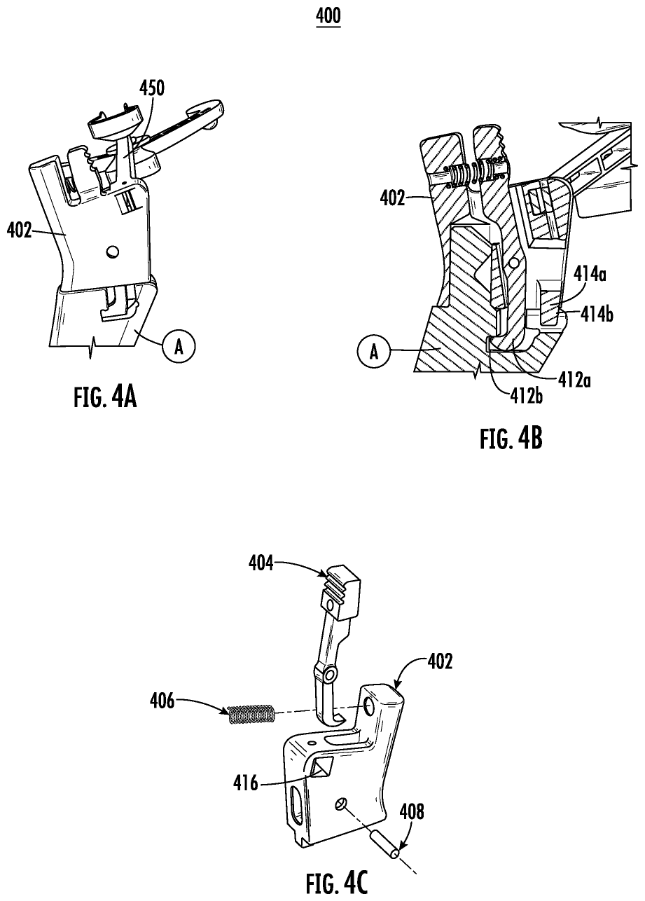

In a first example, broach adapter 400, shown in various views in FIGS. 4A-4C, is provided for attaching the broach array to the top portion of an Anthology broach tool, manufactured by Smith and Nephew, Inc. The overall profile of the attachment tool, the latch mechanism and post hole geometry mimics the neck of the Anthology broach tool, in combination with an anti-rotation tab. This connection completely fixates the broach adapter to the broach tool in situ in the femoral canal.

A side view of broach adapter 400 is shown in FIG. 4A wherein broach adapter 400 is attached to broach “A” which, in this case may be, for example, an Anthology broach tool. A cross-sectional view of broach adapter 400 is shown in FIG. 4B showing the interconnection between broach adapter 400 and broach tool “A”. FIG. 4C shows an exploded view of broach adapter 400. Broach adapter 400 consist of the main body 402 housing a spring-loaded lever 404 pivotally attached to body 402 via pin 408. The lower end of lever 404 includes a latch 412a which engages notch 412b in broach tool “A”. Spring 406 holds hook 412a defined on the end of lever 404 in slot 412b until lever is pivoted about the pivot point to release the broach adapter 400 from broach tool “A”. Spring 406 is retained using a pinned latch (not shown). A blind hole in the body 402 allows broach adapter 400 to be seated against broach tool “A” where latch 412a fits into notch 412b. In addition, a tab 414a engages a slot 414b defined on broach “A” to prevent rotation of broach adapter 400 with respect to broach tool “A”. The broach array holder 450 engages the broach adapter 400 by being inserted into slot 416 using a mechanism that is described later herein.

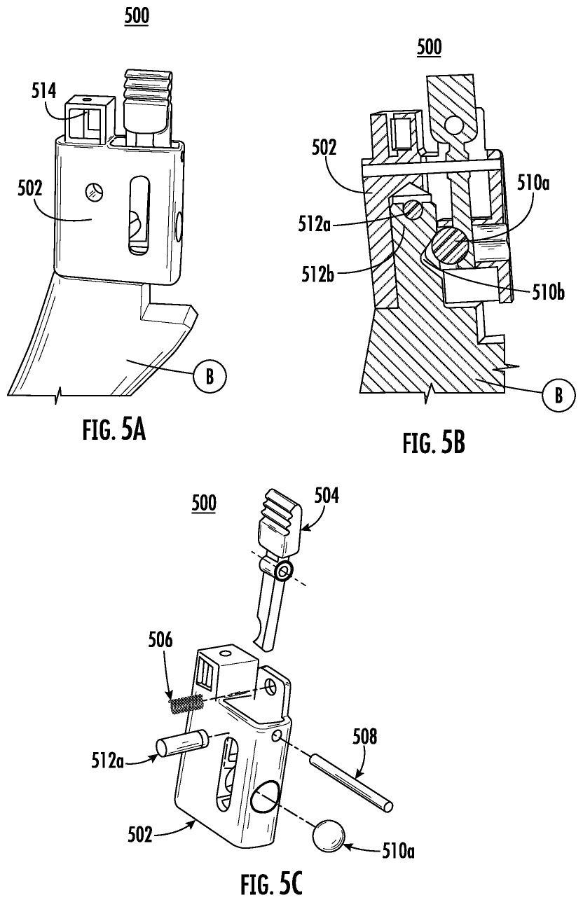

In a second example, broach adapter 500, shown in various views in FIGS. 5A-5C, is provided for attaching the broach array to the top portion of Memphis based stem families including, for example, Synergy, Spectron, previous Anthology broaches, and CPCS broaches. The broach adapter 500 is shown in a side view in FIG. 5A, in cross-sectional view in FIG. 5B and in an exploded view in FIG. 5C.

Broach adapter 500 uses a spring-loaded latch 504 pivotally attached to body 502 via pin 508. Spring-loaded latch 504 captures a ball bearing 510a configured to fit into the notch geometry 510b on broach tool “B”. When spring-loaded latch 504 is actuated, broach tool “B” releases the ball bearing 510a so that it can move away from notch 510b defined on broach tool “B”, allowing removal of the broach adapter 500 from broach tool “B”. A cross pin 512a configured to fit into a U-shaped divot 512b defined on broach tool “B”, in combination with the latch mechanism, constrains the rotation of broach adapter 500 such that it is completely fixed with respect to broach tool “B”. As with the example shown in FIGS. 4A-4C, the broach array holder is configured to be inserted into slot 514, which is identical to slot 416 defined in broach adapter 400.

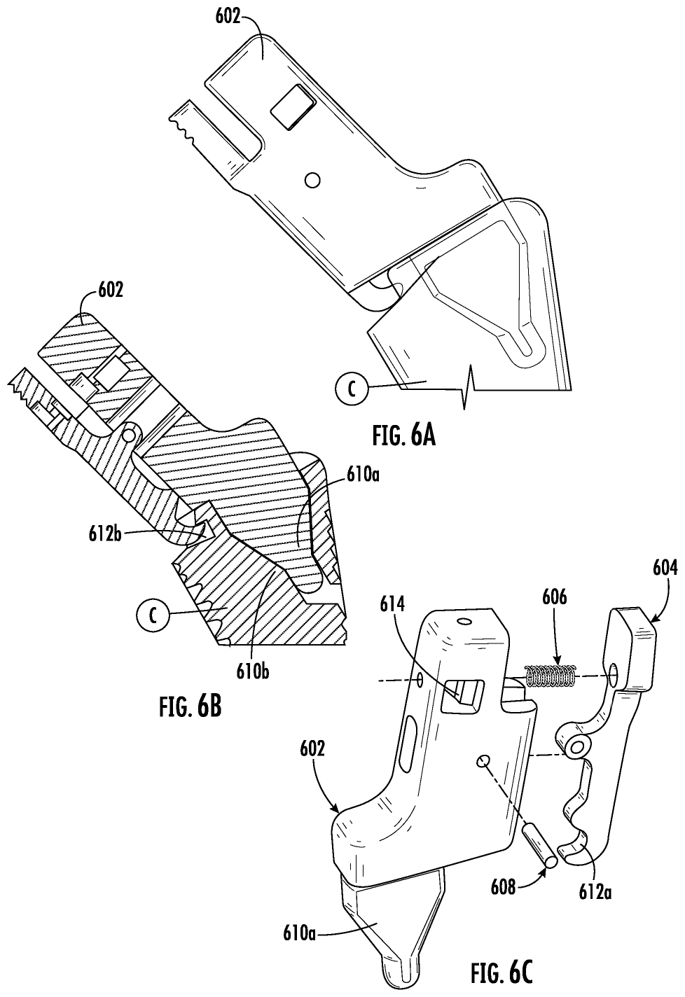

In the third example, broach adapter 600, shown in various views in FIGS. 6A-6C, is provided for attaching the broach array to the top portion of Polarstem, SL Plus, and SL MIA implants and broach tools. The latch and pocket insert geometries mimic the existing Polarstem connection broach geometries that are shared across the three formerly mentioned stem families. A spring-loaded latch and unique pocket insert fully fix the broach adapter to the broach tool. The slot on the medial side will allow for the connection of the array holder shaft.

A side view of broach adapter 600 is shown in FIG. 6A wherein broach adapter 600 is attached to broach “C”. A cross-sectional view of broach adapter 600 is shown in FIG. 6B showing the interconnection between broach adapter 600 and broach “C”. FIG. 6C shows an exploded view of broach adapter 600. Broach adapter 600 consist of the main body 602 housing a spring-loaded lever 604 pivotally attached to body 602 via pin 608. The lower end of lever 604 includes a hook 612a which engages slot 612b in broach tool “C”. Spring 606 is retained in body 602 via a pinned latch and holds hook 612a defined on the end of lever 604 in slot 612b until lever is pivoted about the pivot point to release the broach adapter 600 from broach “C”. A protrusion 610a defined on broach adapter 600 engages a depression 610b defined on broach “C. The broach array holder engages the broach adapter 600 by being inserted into slot 614 using a mechanism that is described later herein.

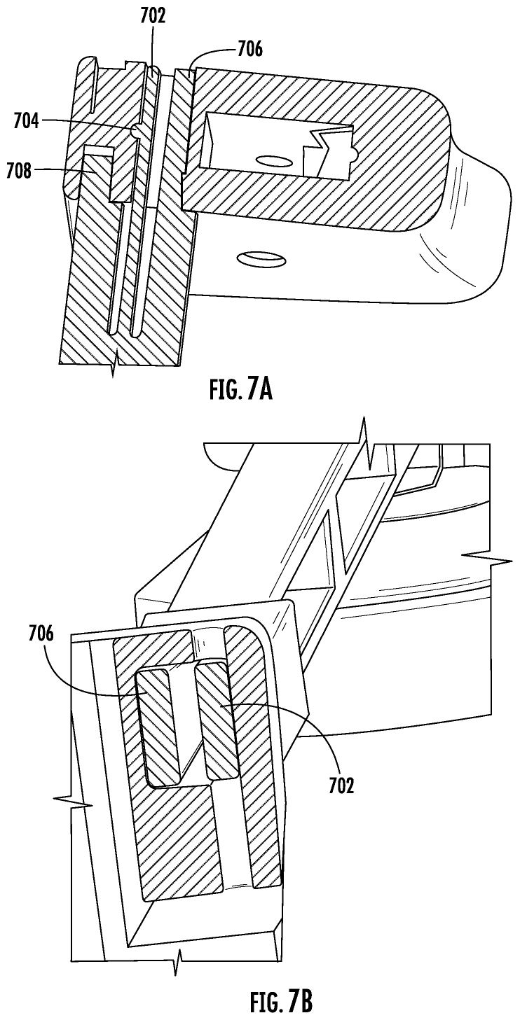

Various examples exhibiting the second feature of the disclosed examples, the broach adapter, have been described. All three examples, as well as any future examples, share a common mechanism for attaching the broach array holder to the broach adapter. This is shown as reference numbers 416, 514 and 614 in FIGS. 4A-4C, 5A-5C and 6A-6C respectively. The mechanism for attaching the broach array holder is shown in top cross-sectional view in FIG. 7A and in side view in FIG. 7B.

The mechanism consist of a thin metal arm 702 having a protrusion 704 defined thereon which engages a depression in the broach adapter. Arms 706 and 708 define shoulders that engage the broach adapter to prevent tilting or rotation of the broach array holder. Protrusion 704 is springingly engaged in the depression in the broach adapter by virtue of arm 702 acting as a spring mechanism. As the broach array holder is disengaged from the broach adapter via a pulling motion, arm 702 moves in the direction of arm 706 such as to disengage protrusion 704 from the depression defined in the broach adapter. Conversely, the broach array holder can be re-engaged to the broach adapter via a pushing motion that causes arm 702 to move in the direction of arm 706 such as to allow the engagement of protrusion 704 in the depression defined in the broach adapter.

In the above detailed description, reference is made to the accompanying drawings, which form a part hereof. In the drawings, similar symbols typically identify similar components, unless context dictates otherwise. The illustrative examples described in the present disclosure are not meant to be limiting. Other examples may be used, and other changes may be made, without departing from the spirit or scope of the subject matter presented herein. It will be readily understood that various features of the present disclosure, as generally described herein, and illustrated in the figures, can be arranged, substituted, combined, separated, and designed in a wide variety of different configurations, all of which are explicitly contemplated herein.

Specific examples of the broach adapter have been described herein. As would be realized by one of skill in the art, different broach tools having different geometries may come to market and will require differently designed broach adapters. However, all broach adapters will be configured with similar characteristics, namely, they will be configured with a common receptacle to accept the broach array holder and being easily removable from the broach tool via a spring mechanism. As would be further realized by one of skill in the art, a specific example of the mechanism for attaching the broach array holder to the broach adapter has been described. However, the disclosed examples are not meant to be limited by the specific examples described herein but is meant to encompass other examples which perform the same function.

Therefore, the present disclosure is not meant to be limited in terms of the particular examples described in this application, which are intended as illustrations of various features. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. It is to be understood that this disclosure is not limited to particular methods, reagents, compounds, compositions or biological systems, which can, of course, vary. It is also to be understood that the terminology used herein is for the purpose of describing particular examples only and is not intended to be limiting.

1. A method for measuring the position of a broach tool in a total hip arthroplasty procedure comprising:
attaching a first tracking array to the femur of a patient;
registering the position of the femur using the tracking array;
attaching a second tracking array to a broach tool after broaching the femoral canal; and
determining the position of the broach tool with respect to the femur based on a comparison between position of the first and second tracking arrays.

2. The method of claim 1, wherein the first tracking array is attached to the femur via a mounting plate that magnetically engages with a mating plate on a holder for the first tracking array.

3. The method of claim 2, wherein the mounting plate allows for multiple orientations of the first tracking array.

4. The method of claim 2, wherein the mounting plate is attached to the proximal femur targeting the greater trochanter.

5. The method of claim 1, wherein:
registering the position of the femur includes tracking the first tracking array using a tracking system having one or more sensors to collect real-time position data; and
software running on a surgical computer calculates and registers the position of the femur based on the position data.

6. The method of claim 1, wherein attaching the second tracking array to the broach tool comprises:
providing a broach adapter attached to the broach tool and attaching the second tracking array to the broach adapter.

7. The method of claim 6, wherein the broach adapter is adapted to fit the geometry of the broach tool.

8. The method of claim 6, wherein the broach adapter attaches to the broach tool using a spring mechanism.

9. The method of claim 6, wherein the broach adapter includes a slot adapted to accept a proximal end of a tracking array holder.

10. The method of claim 9, wherein the proximal end of the tracking array holder includes a spring component having a protrusion thereon which engages a depression defined in the slot of the broaching adapter.

11. The method of claim 1, wherein comparing the position of the first and second tracking arrays comprises:
using a tracking system having one or more sensors to collect real-time position data of the first and second tracking arrays; and
calculating the position of the broach tool relative to the registered position of the femur based on the position data.

12. The method of claim 11, wherein calculating the position of the broach tool is performed by software running on a surgical computer.

13. A device for adapting a holder for a tracking array to a broach tool comprises:
a body shaped to adapt to the geometry of the broach tool;
a lever forced by a spring to engage a feature of the geometry of the broach tool; and
a slot defined in the body of the device for accepting a holder for a tracking array.

14. The device of claim 13, wherein the broach tool is disengaged from the body by pivoting the lever such as to disengage the lever from the feature of the geometry of the broach tool to which it was engaged.

15. The device of claim 13, further comprising:
a holder for a tracking array;
wherein the slot defined in the body is adapted to accept a proximal end of the holder for the tracking array, the proximal end including an arm having a protrusion defined thereon that engages a depression defined in the slot.

16. The device of claim 15, wherein a distal end of the holder for the tracking array includes multiple arms, at least some of the arms defining shoulders to prevent rotation or tilting of the holder with respect to the body.

17. The device of claim 15, wherein the holder for the tracking array is disengaged from the body by bending the arm such that the protrusion disengages from the depression defined in the slot.

18. A system comprising:
a tracking system having one or more sensors;
a processor; and
software for execution on the processor, the software causing the system to:
determine a position of a femur by tracking a first tracking array mechanically connected to the femur using the tracking system to collect real-time position data of the first tracking array; and
determine the position of a broach tool disposed in the femoral canal of the femur by tracking a second tracking array mechanically connected to the broach tool using the tracking system to collect real-time position data of the second tracking array;
wherein the software calculates the position of the broach tool with respect to the femur based on a comparison of the position data representing the position of the first and second tracking arrays.
